(19) United States
(12) Patent Application Publication
(10) Pub. No.: US 2025/0333798 A1
(43) Pub. Date: Oct. 30, 2025

(54) MOLECULAR MARKER RELATED TO BODY WEIGHT TRAITS OF HU SHEEP AND APPLICATION

(71) Applicant: Zhejiang University, Hangzhou (CN)

(72) Inventors: Zhengguang WANG, Hangzhou (CN); Feng QIU, Hangzhou (CN); Jizhong XIANG, Hangzhou (CN); Fengqin PAN, Hangzhou (CN); Guiting WANG, Hangzhou (CN); Jiali TONG, Hangzhou (CN); Zhonghua FEI, Hangzhou (CN); Xin XIANG, Hangzhou (CN); Chong WANG, Hangzhou (CN); Xinming DUAN, Hangzhou (CN)

(73) Assignee: Zhejiang University, Hangzhou (CN)

(21) Appl. No.: 19/090,470

(22) Filed: Mar. 26, 2025

(30) Foreign Application Priority Data
Apr. 26, 2024 (CN) 202410513767.8

(51) Int. Cl. C12Q 1/6888

(52) U.S. Cl. CPC C12Q 1/6888; C12Q 2600/124

(57) ABSTRACT

A molecular marker related to body weight traits of Hu sheep and an application are provided. The whole genome sequencing of Hu sheep is carried out in the invention, the combination of QTL and GWAS is adopted, the candidate genes related to the weight traits are analyzed and the three molecular markers of MAP3K1, ABCB1, and MEF2C are selected as candidate genes for the weight traits of Hu sheep, and the two molecular markers of ANKRD55 and TRNAW-CCA-87 can be used as potential candidate genes for the weight traits of Hu sheep, these candidate genes can be used as molecular markers for application, the guidance of Hu sheep breeding will help to understand the genetic mechanism of weight traits of Hu sheep and further guide Hu sheep breeding.

### CROSS REFERENCE TO THE RELATED APPLICATIONS

This application is based upon and claims priority to Chinese Patent Application No. 202410513767.8, filed on Apr. 26, 2024, the entire contents of which are incorporated herein by reference.

### TECHNICAL FIELD

The invention relates to agricultural genetic engineering, in particular to a molecular marker related to body weight traits of Hu sheep and an application.

### BACKGROUND

Quantitative trait loci (QTL) is a locus associated with phenotypic variation in organisms, which refers to the location of genes controlling quantitative traits in the genome. Since the 1990s, QTL has been playing an important role in the identification of economic traits in livestock, however, QTL usually covers a large number of genomes, resulting in low resolution. Genome-wide association study (GWAS) is a method of research at the genome-wide level. It is characterized by the association analysis of genotype and phenotype, which can identify high-density genetic markers (single nucleotide polymorphism (SNP) or Copy number variation, (CNV), etc.). GWAS and QTL have good complementarity, and GWAS can be used to identify the main QTLs of livestock economic traits. Allais et al. identified QTL maps related to chicken body weight, carcass composition, and meat quality traits. Xu et al. conducted GWAS on the growth and fat traits of Sujiang pigs and found that four genes (GABRB3, ZNF106, XKR4, and MGAM) can be used as candidate genes for body weight traits and backfat thickness traits. Zhang et al. conducted GWAS on the body weight traits of Inner Mongolia Cashmere Goats and found that MAPK3, LDB2, and LRPIB genes were key candidate genes for body weight traits. In addition, RAB6B and GIGYF2 were considered as candidate genes related to the birth weight traits of Lori-Bakhtiari sheep. Li et al. analyzed the growth and feed efficiency of broilers by GWAS and QTL integration analysis, and identified two related QTL areas, and found that NSUN3, EPHA6, and AGK were candidate genes in the related areas.

Hu sheep is a world-famous multi-lamb sheep breed and a first-level local protected breed in China, Hu sheep has the advantages of early sexual maturity, high fecundity, high slaughter rate, and delicious meat, Hu sheep has great potential waiting for us to explore. Therefore, in this study, genome-wide sequencing was performed on 257 Hu sheep individuals, and genome-wide association analysis was performed on the four body weight traits of birth weight, weaning weight, weights at 6 months of age, and weights at 12 months of age to determine the candidate genes related to body weight traits of Hu sheep. The invention discovered and verified candidate genes related to weight traits of Hu sheep, which can be used as molecular markers to provide a basis for genetic improvement of Hu sheep and can be used to guide future breeding decisions.

### SUMMARY

In order to solve the shortcomings of the existing technology, the purpose of the invention is to provide a molecular marker related to the weight traits of Hu sheep and an application, the invention screened three molecular markers, MAP3K1, ABCB1, and MEF2C, which can be used as candidate genes for the weight traits of Hu sheep, and screened two molecular markers, ANKRD55 and TRNAW-CCA-87, which can be used as potential candidate genes for the weight traits of Hu sheep.

In order to achieve the above objectives, the invention adopts the following technical scheme:

A molecular marker related to body weight traits of Hu sheep, Mitogen-Activated Protein 3 Kinase 1 (MAP3K1), ATP-binding cassette subfamily B member 1 (ABCB1) or MEF2C is a molecular marker related to the body weight traits of Hu sheep.

A molecular marker related to body weight traits of Hu sheep, ANKRD55 or TRNAW-CCA-87 is a molecular marker related to the weight traits of Hu sheep.

An application of the aforementioned molecular marker is applied to an establishment of a core breeding group of Hu sheep.

An application of the aforementioned molecular marker is applied to a cultivation of new strains.

An application of the aforementioned molecular marker is applied to a 50K chip dedicated to Hu sheep, a content of the 50K chip is based on the molecular marker and combined with a candidate gene for other economic traits of Hu sheep.

The benefits of the invention lie in:

Three molecular markers, MAP3K1, ABCB1, and MEF2C, are screened out as candidate genes for body weight traits of Hu sheep, and two molecular markers, ANKRD55 and TRNAW-CCA-87, are screened out as potential candidate genes for body weight traits of Hu sheep.

The invention verifies that the candidate genes are closely related to the body weight traits of Hu sheep, these molecular markers have a wide range of applications, for example, they can be applied to the formation of the core breeding group of Hu sheep, or to the cultivation of new lines, and can also be applied to the special 50 K chip of Hu sheep to guide the breeding of Hu sheep.

### BRIEF DESCRIPTION OF THE DRAWINGS

FIGS. 1A-1E are genome-wide association analysis correlation diagram of the birth weight trait of the invention; (FIG. 1A: Manhattan diagram of the genome-wide association analysis of birth weight traits; FIG. 1B: Quantile-quantile (Q-Q) plot of the genome-wide association results of birth weight traits; FIG. 1C: QTL annotation map (exterior, health, meat and carcass, milk, production, reproduction, wool); FIG. 1D: Percentage of each trait marked as producing QTL in the candidate interval (kleiber ratio, bone density, body height, average daily gain, body weight); FIG. 1E: QTL enrichment analysis (trichostrongylus colubriformis FEC, trichostrongylus adult and larva count, teatplacement, staple strength, reproductive seasonality, primary fiber diameter coefficient of variance, milk fat percentage, meat color, lean meat yield percentage, kleiberratio, internal fat amount, hot carcass weight, hematocrit, Haemonchus contortus FEC, fecal egg count, dressing percentage, change in hematocrit, bone weight in carcass, body weight, backfat at third lumbar), the richness factor is obtained by the ratio of the number of QTLs annotated in the candidate area to each total number of QTLs.

FIGS. 2A-2E are genome-wide association analysis correlation diagram of the weaning weight trait of the invention; (FIG. 2A: Manhattan diagram of the genome-wide association analysis of weaning weight traits; FIG. 2B: Q-Q plot of the genome-wide association results of weaning weight traits; FIG. 2C: QTL annotation map; FIG. 2D: Percentage of each trait labeled as a production QTL in the candidate interval (residual feed intake, total bone, body weight); FIG. 2E: QTL enrichment analysis (useful yield content, uddershape, total bone, testes weight, somatic cell count, salmonella abortusovis susceptibility, residual feed intake, muscle weight in carcass, milk fat percentage, milk bacterial count, meat color L*, meat color b*, meat color a*, longissimus muscle area, lean meat yield percentage, hot carass weight, fat weight in carcass, facial eczema susceptibility, dressing percentage, carcass fat percentage), the richness factor is obtained by the ratio of the number of QTLs annotated in the candidate area to each total number of QTLs.

FIGS. 3A-3E are genome-wide association analysis correlation diagram of body weight traits at 6 months of age of the invention; (FIG. 3A: Manhattan diagram of the genome-wide association analysis of body weight traits at 6 months of age; FIG. 3B: Quantile-quantile (Q-Q) plot of the genome-wide association results of body weight traits at 6 months of age; FIG. 3C: QTL annotation map; FIG. 3D: Percentage of each trait in the candidate interval as a production QTL marker (age at maximum daily gain, total bone, bone density, body weight); FIG. 3E: QTL enrichment analysis, the richness factor is obtained by the ratio of the number of QTLs annotated in the candidate area to each total number of QTLs (testes weight, somatic cell count, shear force, primary fiber diameter coefficient of variance, muscle depth at third lumbar, milk fat yield, milk fat percentage, meat palmitoleic acid content, meat linolenic acid content, meat eicosapentaenoic acid content, meat docosapentaenoic acid content, meat arachidonic acid content, lean meat yield percentage, fat weight incarcass, fleece yield, bone weight in carcass, body weight age at maximum daily gain);

FIGS. 4A-4E are genome-wide association analysis correlation diagram of body weight traits at 6 months of age of the invention; (FIG. 4A: Manhattan map for genome-wide association analysis of body weight traits at 12 months of age; FIG. 4B: Quantile-quantile (Q-Q) map of genome-wide association results for weight traits at 12 months of age; FIG. 4C: QTL annotation map; FIG. 4D: Percentage of each trait in the candidate interval as a production QTL marker (bone density, body weight); FIG. 4E: QTL enrichment analysis, the richness factor is obtained by the ratio of the number of QTLs annotated in the candidate area to each total number of QTLs (wool crimp, trichuostrongylus colubriformis FEC, Trichuostrongylus adult and larva count, staple length, scrapie susceptibility, salmonella abortusovis susceptibility, primary fiber diameter coefficient of variance, muscle depth at third lumbar, milk pentadecylic acid content, milk palmitic acid content, muscle weight in carcass, milk fat percentage, milk cis-10heptadecic acid content Milk conjugated linoleic acid content, meat color L*, meat color b*, meat color a*, longissimus muscle area, lean meat yield percentage, hot carass weight, fecal oocyst count, fat weight in carcass, entropion);

FIGS. 5A-5D: box plot showing different phenotypic differences according to genotype.

### DETAILED DESCRIPTION OF THE EMBODIMENTS

The following is a specific introduction to the invention in combination with the attached figures and specific embodiment.

The following experiments show that the three molecular markers MAP3K1, ABCB1, and MEF2C can be used as candidate genes for body weight traits in Hu sheep, and the two molecular markers ANKRD55 and TRNAW-CCA-87 can be used as potential candidate genes for body weight traits in Hu sheep.

### 1. Materials and Methods

### 1.1 Phenotypic Measurement and Sample Preparation

Hu sheep (n=257) used in this study were all from Huzhou Yihui Ecological Agriculture Co., Ltd. All Hu sheep were randomly selected and raised in the same environment. The birth weights, weaning weights, weights at 6 months of age, and weights at 12 months of age of Hu sheep were measured using an electronic scale in a relaxed and quiet environment. Blood samples were collected from the posterior vein of Hu sheep and stored in a −80° refrigerator to prepare for DNA extraction. We used the CWE9600Magbead Blood DNA Kit and magnetic bead method to extract DNA. The extracted DNA samples were spotted in 1.5% agarose gel to detect the integrity of DNA samples; then the purity of DNA was detected by NanoDrop 2000 nucleic acid protein analyzer; finally, dsDNA HS Assay Kit for Qubit was used to accurately quantify the samples.

### 1.2 Library Construction and Sequencing

In the process of library construction, a universal library construction kit (for MGI) DNA was used. The genomic DNA with a total amount of more than lug was randomly disrupted into fragments of about 300-350 bp by Covaris TM, after end repair, A tail was added and the sequencing adaptor was ligated, SP Beads was used to screen DNA of about 300-350 bp for PCR amplification, and SP Beads was used again to purify the PCR product, and finally the sequencing library was obtained. After the library construction was completed, Qubit2.0 was used for preliminary quantification, and then the insert fragments of the library were detected using (Agilent). After the library inspection was qualified, pooling was performed according to the effective concentration of the library and the amount of target offline data, the DNBSEQ-T7 sequencer was used to select the sequencing test of PE150 for on-machine sequencing.

### 1.3 Genotyping and Quality Control

The original data was subjected to quality control steps such as de-jointing and filtering to obtain clean data, which is used for subsequent bioinformatics analysis. In order to ensure the quality of information analysis, fastp is used to perform a series of quality control on raw reads. After data filtering, the reference genome Ovisaries (assembly ARS-UI_Ramb_v2.0) was indexed, and then Clean Reads was aligned to the reference genome by BWA0.7.17 (men) software, samtools 1.7 was sorted and indexed, the Bam file was deduplicated using the module included in GATK 4.1.8.0 software, and then the sequencing depth and genome coverage of each sample were counted based on the Bam file to prepare for subsequent mutation detection. According to the alignment results of Clean Reads in the reference genome, we used the software GATK 4.1.8.0 callSNP, and then the VariantFiltration module was used to strictly filter SNP and INEDL. The specific filtering criteria for SNPs were to remove the loci with >10% deletions, the loci when the minimum allele frequency was less than 0.05, and the loci when the Hardy-Weinberg test P value was less than 0.000001. For the missing SNP loci, we used Beagle5.2 for filling. Because the threshold calculated by the Bonferroni method (0.05/SNP number) is too strict, independent SNPs are extracted in this time by the parameters of PLINK's-indep-pairwise100100.2, and the significance threshold is calculated by 1/independent SNP number (1/710129), that is, p<1.41E-06.

### 1.4 Genome-Wide Association Analysis

GWAS analysis was performed using GMAT software. The model used in GWAS analysis is a mixed linear model, which can well correct the population structure and complex genetic relationships within the population. The formula of the model is as follows:

$$y = X β + Z k γ k + ξ + e$$

wherein y is a phenotypic vector, Xβ is the population structure effect and the fixed effect of sex, birth season, and measured month age, Zkγk is the marker effect to be tested (multi-gene effect), and e-N(0, Iσ2) is a residual effect. K in the polygenic effect is the genetic relationship matrix inferred by markers. Then, the qqman package of R software was used to draw the Manhattan diagram and Q-Q diagram. Haplotype analysis was performed using Haploview software for the selected significant SNPs, and the range of selection was all SNP sites in the 5 kb interval between the upstream and downstream of the significant SNP. Based on this, a Block with D′ greater than 0.8 was used as a haplotype block.

### 1.5 Gene Annotation and Enrichment Analysis of Significant SNP Loci

The reference genome information Ovis aries (assembly ARS-UI_Ramb_v2.0) of sheep was downloaded on the ENSEMBL website, and significant SNPs were annotated to their corresponding genes using ANNOVAR software. Then, the clusterProfiler package of R software was used to analyze the gene function enrichment of the annotated candidate genes according to the GO and KEGG databases. The animal QTL database (www.animalgenome.org/QTLdb)) was used to annotate the potential functions of the selected areas. We used the GALLO package of R software to perform QTL enrichment analysis, and the screening threshold for significantly enriched QTLs was p-value<0.05. Then, we performed a combination of GWAS and QTL analysis to screen candidate genes associated with body weight traits in Hu sheep.

### 2. Results

### 2.1 Quality Control and Data Statistics

After quality control, a total of 23897134 SNPs were obtained, after screening, 239 significant SNPs and 122 candidate genes (TableS1) were obtained. A total of 257 Hu sheep were selected and their body weight traits, including birth weights, weaning weights, weights at 6 months of age, and weights at 12 months of age, were counted. After excluding unqualified individuals, a descriptive analysis of the phenotype was performed to provide a preliminary analysis of the overall characteristics. Table 1 shows the weight traits of the Hu sheep population.

TABLE 1
Descriptive statistics of body weight
traits of the Hu sheep population

| | Sample | Mean | | CV | Minimum | Maximum |
|---|---|---|---|---|---|---|
| Phenotype | size | value | SD | (%) | value | value |
| Birth weight | 245 | 2.71 | 0.17 | 6.39 | 2.40 | 3.25 |
| Weaning | 251 | 16.21 | 1.03 | 6.37 | 13.70 | 19.80 |
| weight | | | | | | |
| Weight at 6 | 232 | 31.02 | 1.29 | 4.15 | 28.20 | 34.20 |
| months of age | | | | | | |
| Weight at 12 | 241 | 47.73 | 2.53 | 5.30 | 41.40 | 56.30 |
| months of age | | | | | | |

### 2.2 Genome-Wide Association Analysis of Birth Weight

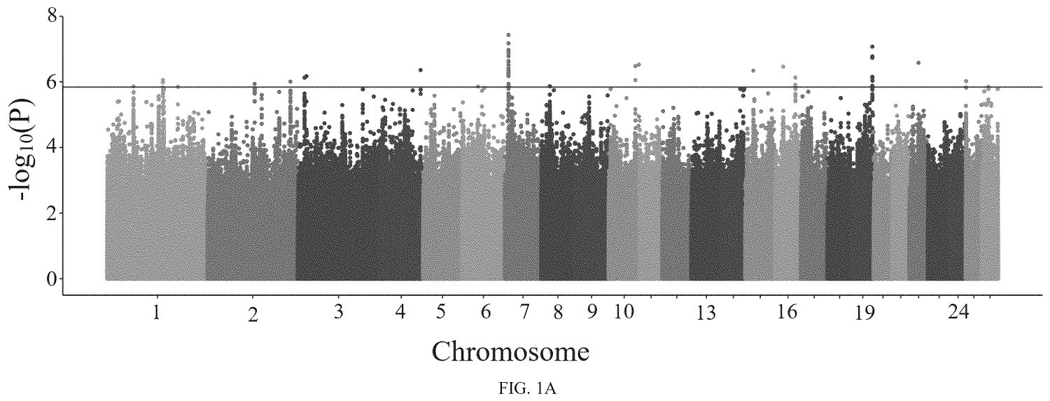

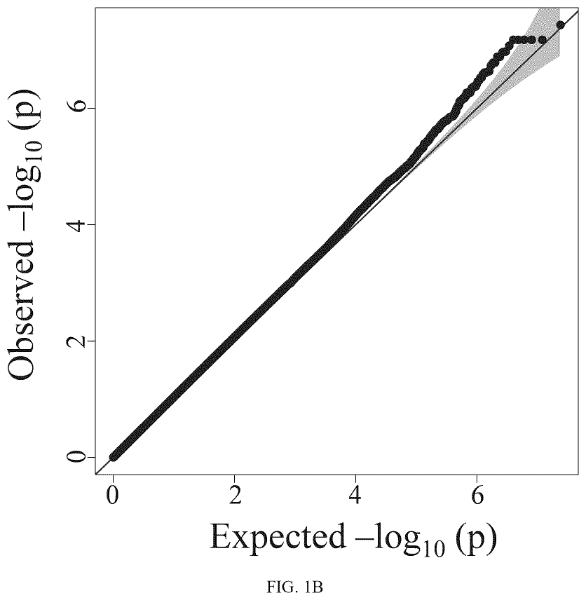

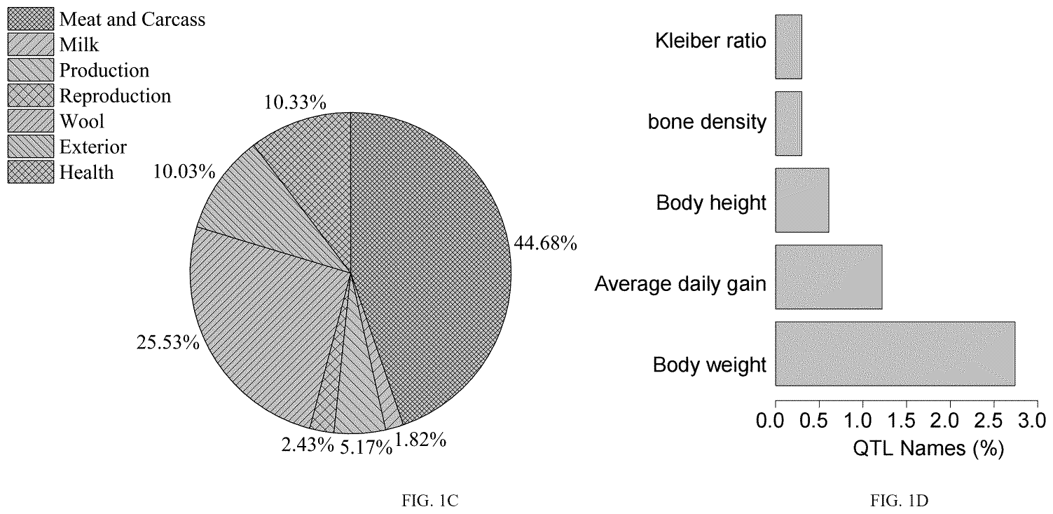

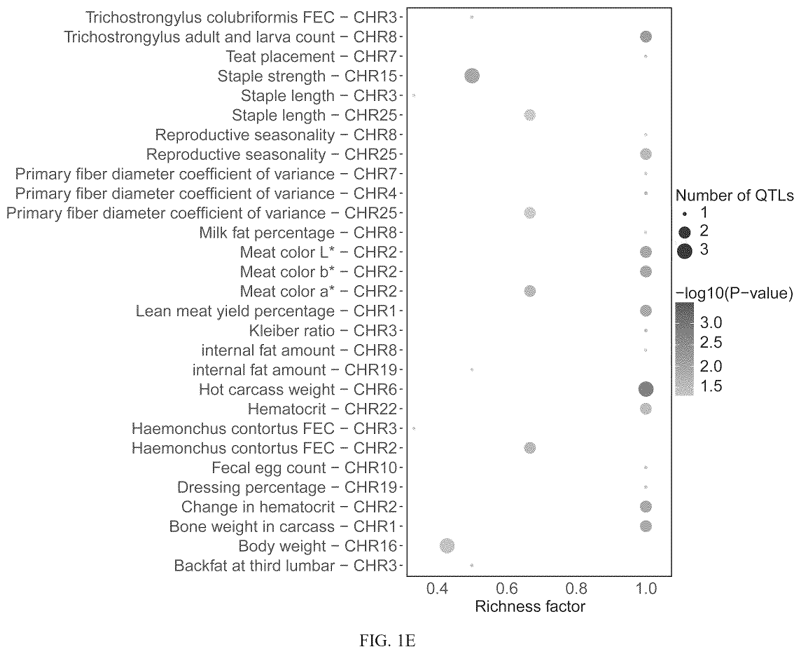

For the birth weight trait, GWAS analysis shows that there are 67 SNPs significantly associated with the birth weight trait, which are distributed on 14 chromosomes, at the same time, we screened 34 candidate genes (TableS1, FIG. 1A). FIG. 1B is the Q-Q plot of the phenotypic trait of birth weight. The deviation between the observed value and the expected value indicates that our model is reasonable and the loci significantly associated with the trait are found. FIG. 1C is the QTL annotation map, meat and carcass QTL accounts for the largest proportion (44.68%), followed by wool (25.53%), health status (10.33%), exterior (10.03%), production (5.17%), reproduction (2.43%), milk production (1.82%). QTL enrichment analysis shows that a total of 329 QTLs are enriched, where 29 QTLs are significantly enriched. Most QTLs are associated with meat, carcass, and wool (TableS2, S3). In the production category, the weight QTL is the most enriched QTL, and we note that the weight QTL is significantly enriched on chromosome 16 (FIG. 1D, FIG. 1E). On chromosome 16, SNP16_22650858 is significantly enriched in weight QTL, and SNP16_22650858 is significantly enriched in gene MAP3K1 (distance=52428 kb) and ANKRD55 (distance=514199). Therefore, we use MAP3K1 and ANKRD55 as candidate genes for weight traits in Hu sheep.

### 2.3 Genome-Wide Association Analysis of Weaning Weight

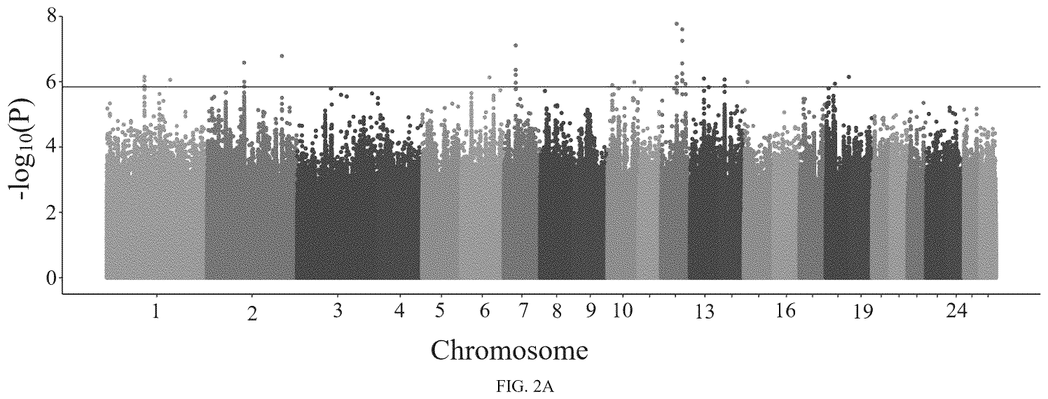

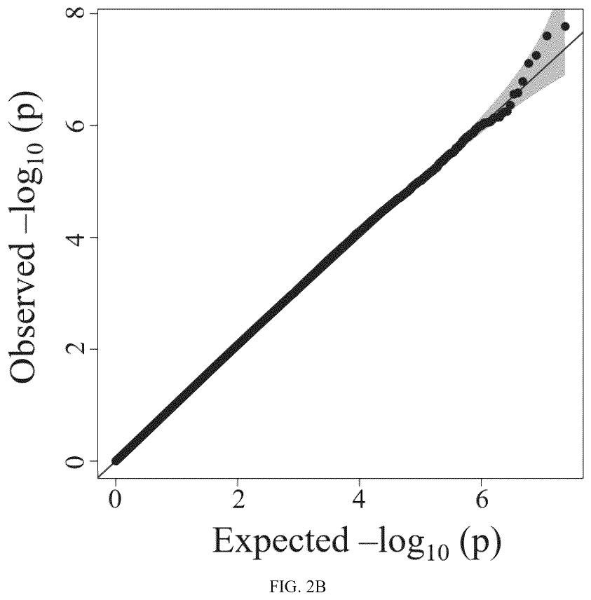

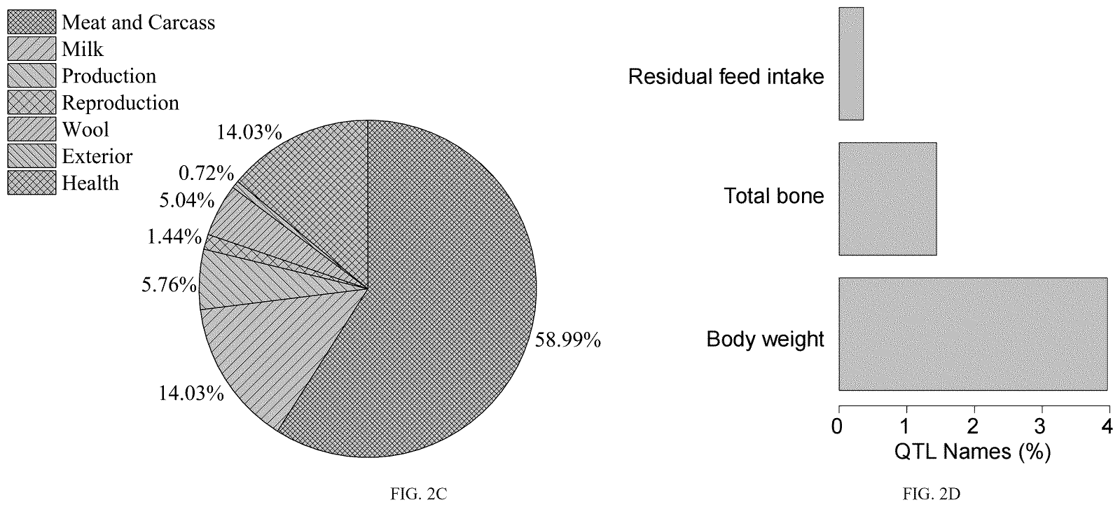

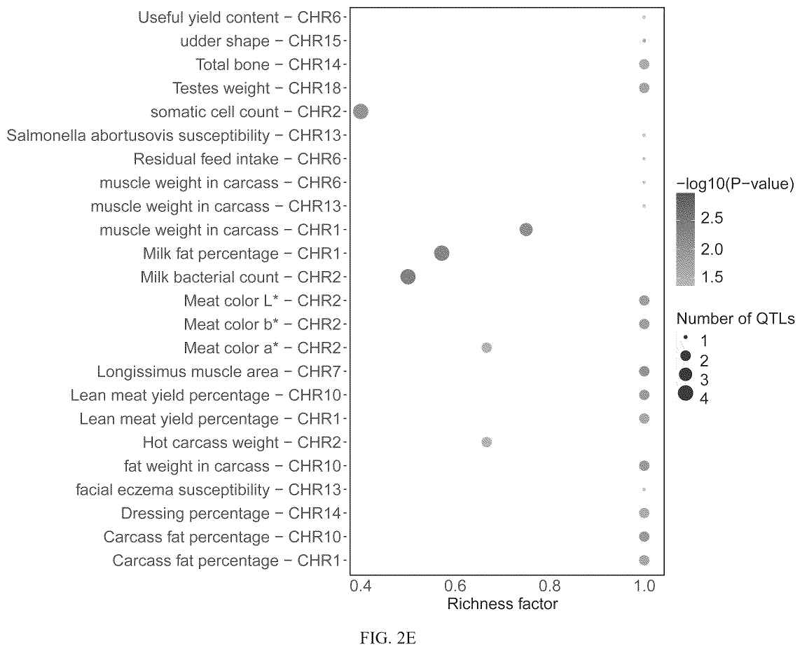

For weaning weight traits, GWAS analysis shows that there are 36 SNPs significantly associated with weaning weight traits, distributed on 10 chromosomes, and we screened 25 candidate genes (FIG. 2A). FIG. 2B is the Q-Q plot of the phenotypic traits of weaning weight, the observed values are basically the same as the expected values, indicating that our model is reasonable and has certain loci significantly associated with the traits. FIG. 2C is the QTL annotation map, meat and carcass QTL account for the largest proportion (58.99%), followed by health status and milk production (both 14.03%), production (5.76%), wool (5.04%), reproduction (1.44%), exterior (0.72%). QTL enrichment analysis shows that a total of 278 QTLs are enriched, where 24 QTLs are significantly enriched; most QTLs are related to meat quality (TableS2, S3). Although the body weight QTL is not significantly enriched, it appears in the production QTL category and is the most enriched production QTL, which has a certain guiding effect on our subsequent analysis (FIGS. 2D-2E).

### 2.4 Genome-Wide Association Analysis of Body Weight at 6 Months of Age.

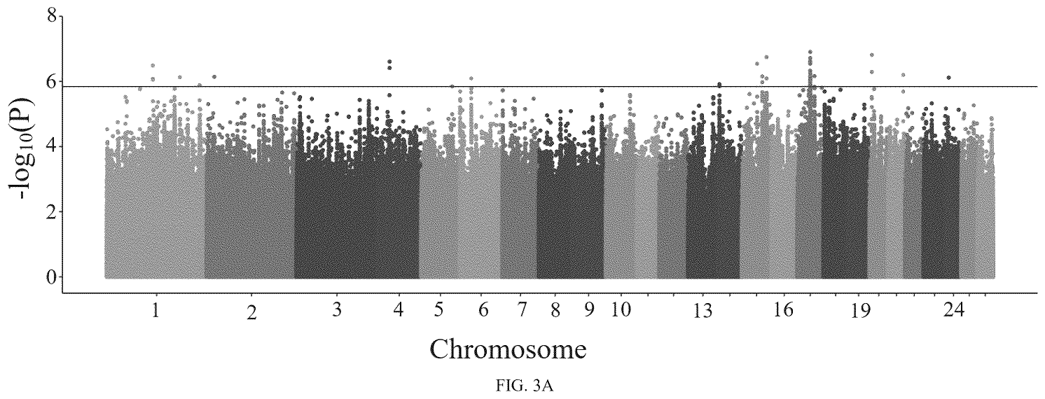

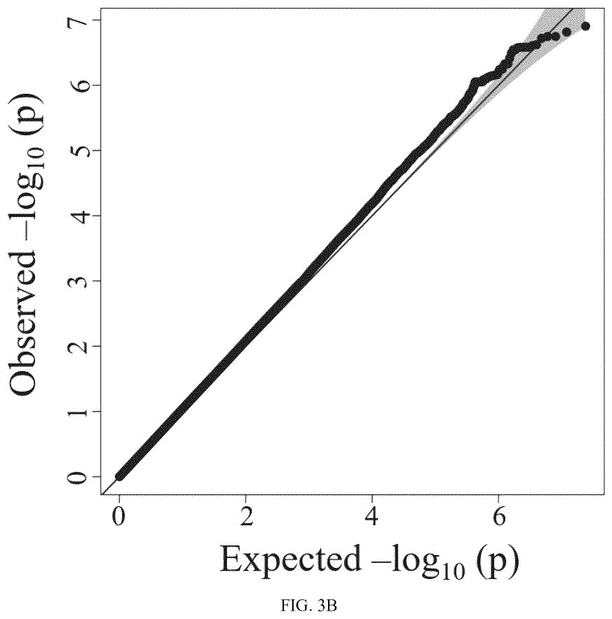

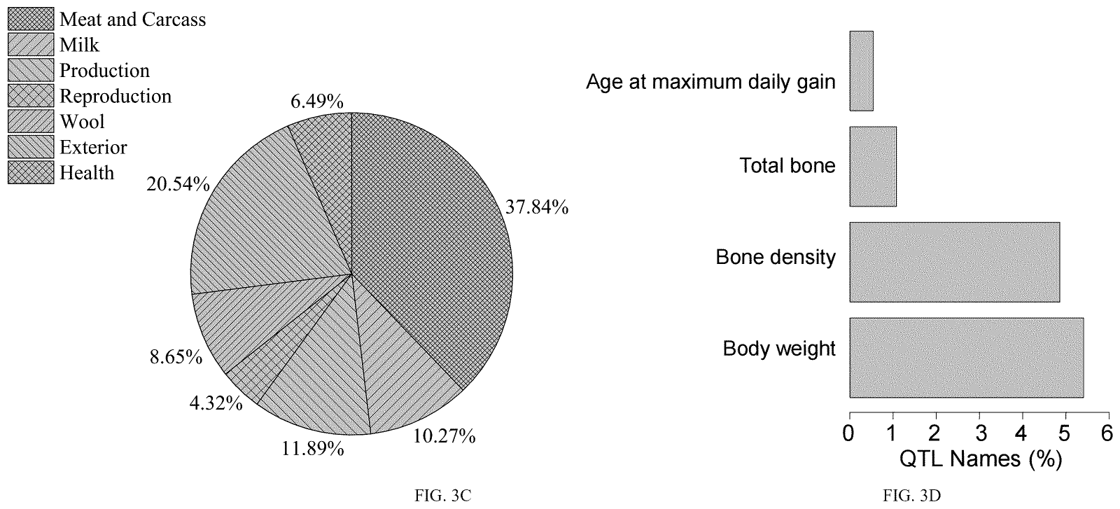

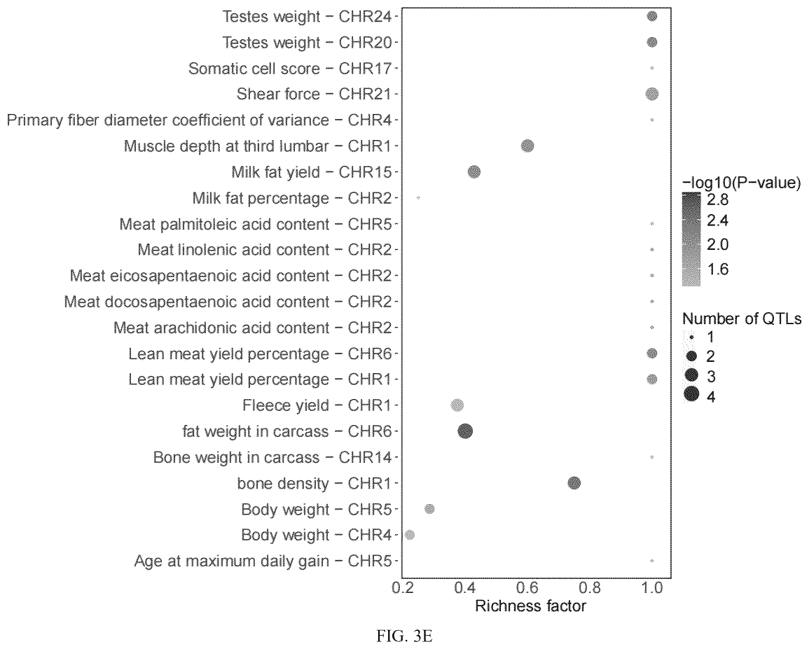

For body weight traits at 6 months of age, GWAS analysis shows that there are 70 SNPs significantly associated with body weight traits at 6 months of age, distributed on 11 chromosomes, and we screened 25 candidate genes (FIG. 3A). FIG. 3B is the Q-Q plot of the body weight phenotypic traits at 6 months of age, the observed values are basically the same as the expected values, indicating that our model is reasonable and has certain loci significantly associated with the traits. FIG. 3C is a QTL annotation map, meat and carcass QTLs account for the most (37.84%), followed by exterior (20.54%), production (11.89%), milk production (10.27%), wool (8.65%), health status (6.49%), and reproduction (4.32%). QTL enrichment analysis shows that a total of 185 QTLs are enriched, where 22 QTLs are significantly enriched; most QTLs are associated with meat, carcass, and production. In the production category, the weight QTL is the most enriched QTL, and at the same time, we note that the weight QTL is significantly enriched on chromosome 4 and chromosome 5. On chromosome 4, SNP 4_33994498 and SNP 4_33994500 are significantly enriched in the weight QTL, and both of them are significantly enriched in the gene ABCB1. On chromosome 5, SNP 5_86689911 is significantly enriched in weight QTL and maximum daily gain age QTL. At the same time, SNP 5_86689911 is significantly enriched in genes MEF2C (distance=613777) and TRNAW-CCA-87 (distance=178413). Therefore, ABCB1, MEF2C, and TRNAW-CCA-87 are selected as candidate genes for body weight traits in Hu sheep.

### 2.5. Genome-Wide Association Analysis of Body Weight at 12 Months of Age

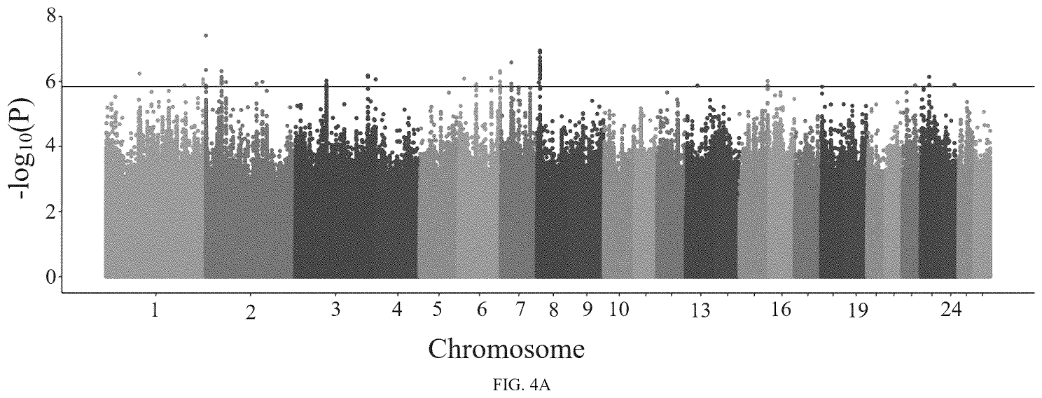

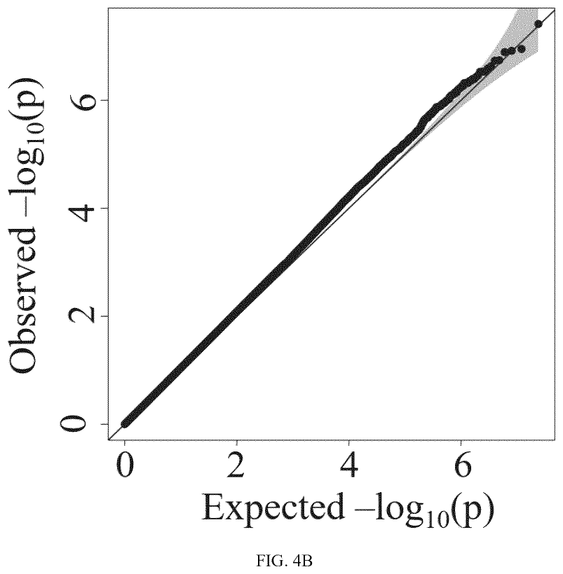

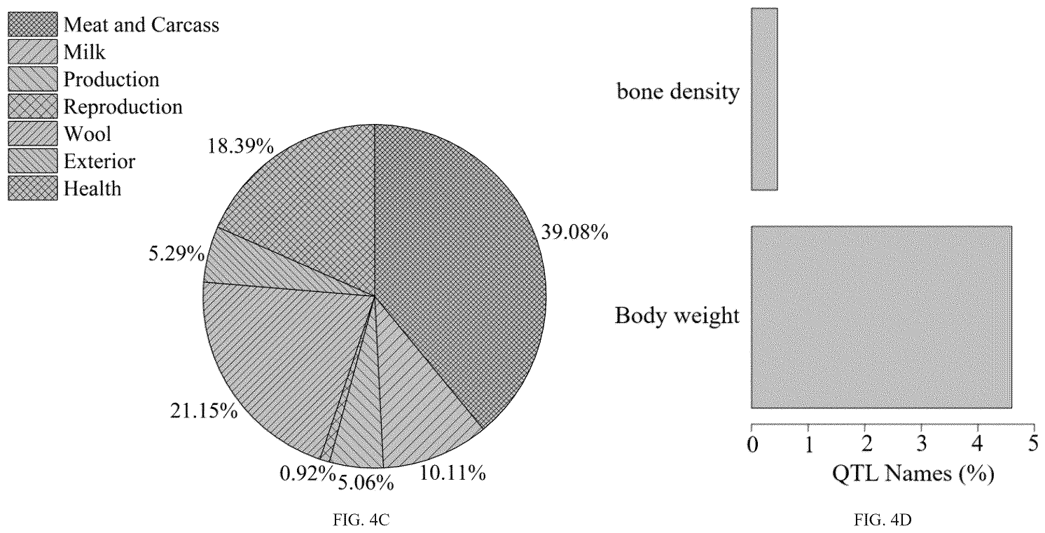

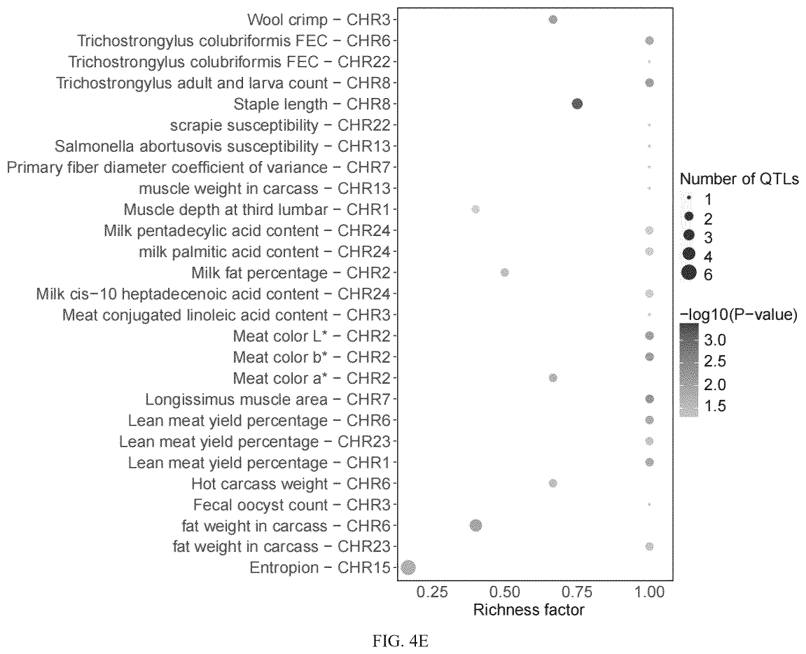

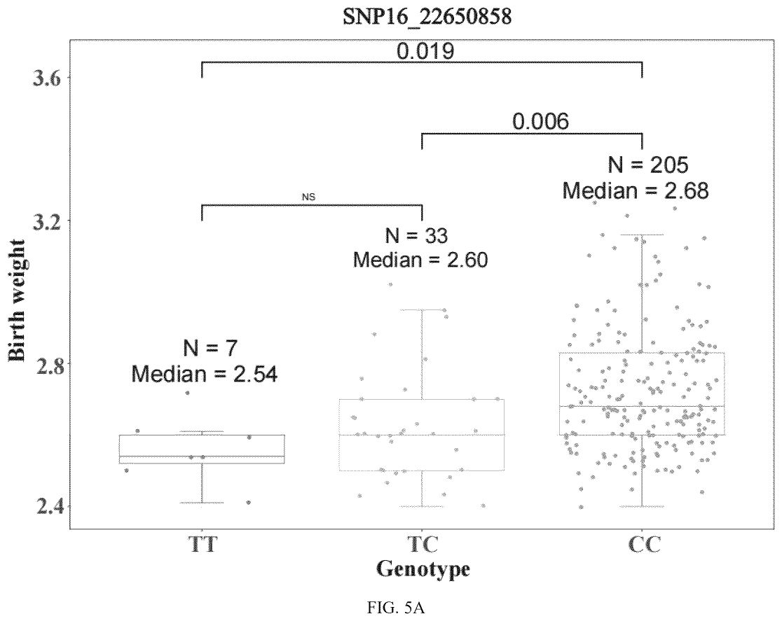

For body weight traits at 12 months of age, GWAS analysis shows that 66 SNPs are significantly associated with body weight traits at 12 months of age, distributed on 11 chromosomes, and 38 candidate genes are screened (TableS1, FIG. 4A). FIG. 4B is the Q-Q plot of the body weight phenotypic traits at 12 months of age. The observed values are basically the same as the expected values, indicating that the model is reasonable and there are some loci significantly associated with the traits. FIG. 4C is the QTL annotation map, meat and carcass QTL account for the largest proportion (39.08%), followed by wool (21.15%), health status (18.39%), milk production (10.11%), exterior (5.29%), production (5.06%), reproduction (0.92%). QTL enrichment analysis shows that a total of 435 QTLs are enriched, where 27 QTLs are significantly enriched. Most of the QTLs are associated with meat, carcass, and health status (TableS2, S3). Although the body weight QTL is not significantly enriched, it appears in the production QTL category and is the most enriched production QTL, which has a certain guiding role for our subsequent analysis (FIGS. 4D-4E).

The significant single nucleotide polymorphisms (SNPs) and candidate genes identified by genome-wide association analysis and QTL are shown in Table 2:

TABLE 2
Significant SNPs and candidate genes identified by combining
genome-wide association analysis and QTL analysis

| | | Position | | | | Related QTL |
|---|---|---|---|---|---|---|
| Phenotype | Chromosome | (bp) | SNP | P-value | Gene/Distance | enrichment |
| Birth | 16 | 22650858 | SNP | 3.43E−07 | MAP3K1/52428; | Weight |
| weight | | | 16_22650858 | | ANKRD55/514199 | |
| Weight at | 4 | 33994498 | SNP | 3.86E−07 | ABCB1/within | Weight |
| 6 months | | | 4_33994498 | | | |
| of age | 4 | 33994500 | SNP | 2.47E−07 | ABCB1/within | Weight |
| | | | 4_33994500 | | | |
| | 5 | 86689911 | SNP5_86689911 | 1.42E−06 | MEF2C/613777; | Weight, age at |
| | | | | | TRNAW-CCA-87/ | maximum daily |
| | | | | | 178413 | gain |

### 2.6 Haplotype Analysis of Significant SNPs

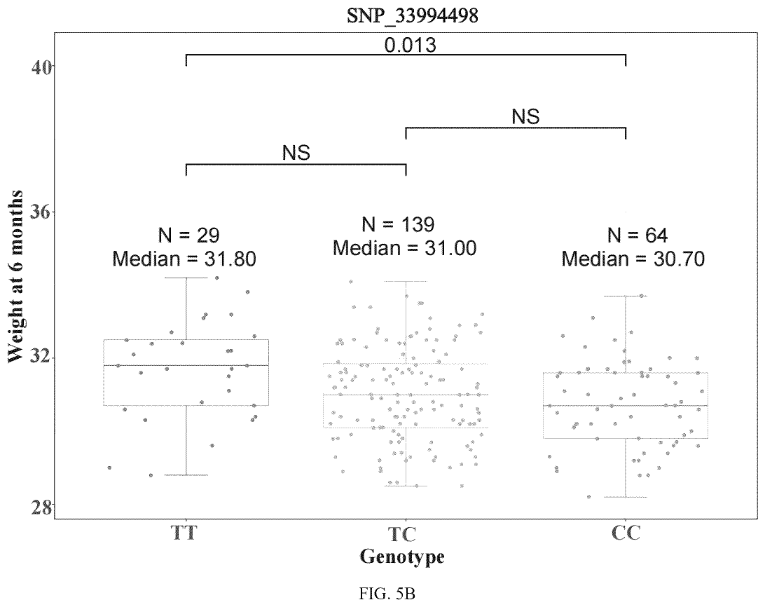

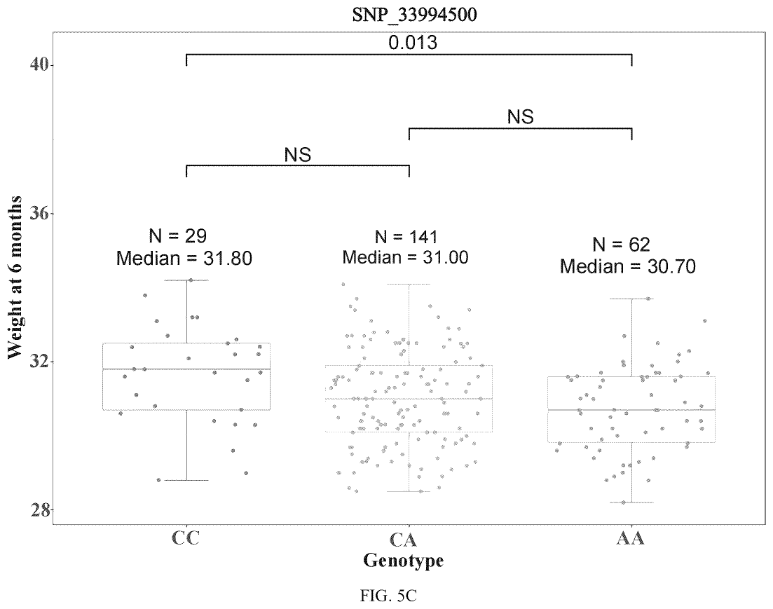

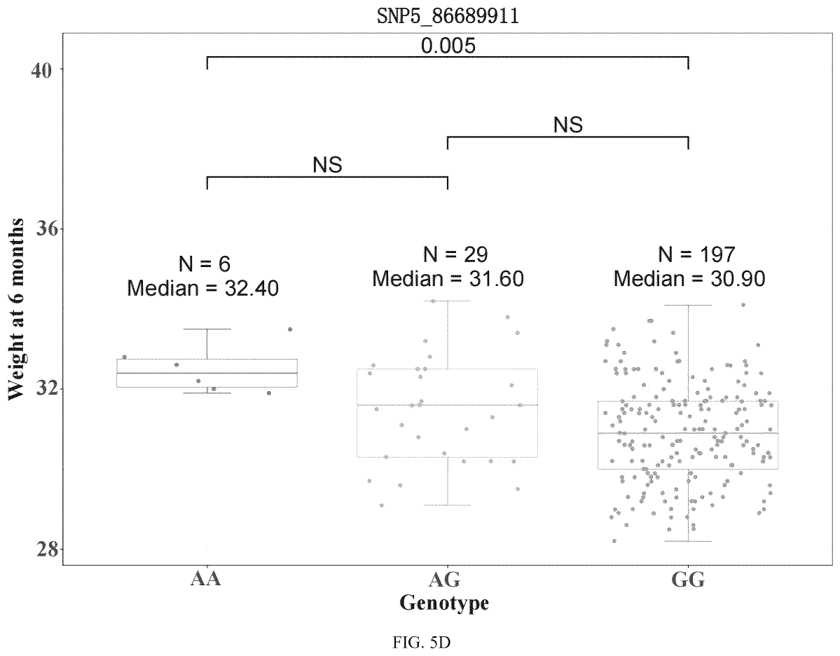

Haploview software is used to analyze the haplotypes of the four significant SNPs (SNP 16_22650858, SNP 4_33994498, SNP 4_33994500, and SNP5_86689911). There is a highly linked Block near these four significant SNPs, and the D′ between all SNPs is greater than 0.8, indicating that the significant SNPs we screened are reasonable. At the same time, we use the KW test to count the phenotypic values of these four significant SNP loci genotypes (FIGS. 5A-5D). Among the four significant SNP loci, there are significant differences between homozygous genotypes, which may be due to the large difference in the number of individuals between the two homozygous genotypes.

### 2.7 GO and KEGG Pathway Enrichment Analysis

In order to further analyze the body weight phenotype of Hu sheep, we perform GO and KEGG pathway enrichment analysis on the candidate genes of the four phenotypes. Gene Ontology (GO) is a comprehensive database describing gene function, which can be divided into three parts: Biological Process (BP), and Cellular Component (CC), Molecular Function (MF). KEGG (Kyoto Encyclopedia of Genes and Genomes) is a comprehensive database that integrates genomic, chemical, and systematic functional information. Among the birth weight traits, 113 GO pathways are significantly enriched, and 13 KEGG pathways are significantly enriched; among the weaning weight traits, 37 GO pathways are significantly enriched, and 3 KEGG pathways are significantly enriched; among the body weight traits at 6 months of age, 35 GO pathways are significantly enriched, and 11 KEGG pathways are significantly enriched; among the body weight traits at 12 months of age, 78 GO pathways are significantly enriched, and no KEGG pathway is significantly enriched. By analyzing the significantly enriched pathways of GO and KEGG, it is found that the significantly enriched pathway of gene MAP3K1 is growth hormone synthesis and secretion. The significantly enriched pathways of gene ABCB1 are ATP binding, gastric cancer, and microRNA in cancer, the significantly enriched pathways of MEF2C are Apelin signaling pathway, oxytocin signaling pathway, and MAPK signaling pathway. There are no significantly enriched pathways in ANKRD55 and TRNAW-CCA-87 (Table 3). The indicative list of candidate genes and their related pathways related to body weight traits of Hu sheep is shown in Table 3.

TABLE 3
Indicative list of candidate genes and related pathways
related to body weight traits in Hu sheep

| | | | Significantly enriched |
|---|---|---|---|
| Phenotype | Chromosome | Gene | pathways |
| Birth | 16 | MAP3K1 | Synthesis, secretion, and |
| Weight | | | function of growth |
| | | | hormone |
| | 16 | ANKRD55 | |
| Weight at 6 | 4 | ABCB1 | RNAATP binding; |
| months of | | | gastric cancer; |
| age | | | microRNAs in cancer |
| | 5 | MEF2C | Apelin signaling |
| | | | pathway; oxytocin |
| | | | signaling pathway; |
| | | | MAPK signaling |
| | | | pathway |
| | 5 | TRNAW-CCA-87 | |

In summary: In this study, the whole genome sequencing of Hu sheep is carried out, and the candidate genes related to body weight traits are analyzed by combining QTL and GWAS. A total of 239 significant SNPs and 122 candidate genes are identified in 257 Hu sheep populations. MAP3K1, ABCB1, and MEF2C can be used as candidate genes for body weight traits of Hu sheep, and ANKRD55 and TRNAW-CCA-87 can be used as potential candidate genes for body weight traits of Hu sheep. The candidate genes of this invention are closely related to the weight traits of Hu sheep, and can be used as molecular markers for application. For example, it can be used to establish the core breeding group of Hu sheep, or to cultivate new strains, and can also be applied to the 50 K chip dedicated to Hu sheep, guiding Hu sheep breeding will help to understand the genetic mechanism of weight traits of Hu sheep and further guide Hu sheep breeding.

The above shows and describes the basic principle, main characteristics, and advantages of the invention. Technicians in this industry should understand that the above-mentioned embodiments do not restrict the invention in any way, and the technical solutions obtained by equivalent substitution or equivalent transformation fall within the protection scope of the invention.

What is claimed is:

1. A molecular marker related to body weight traits of Hu sheep, wherein MAP3K1, ABCB1, or MEF2C is the molecular marker related to the body weight traits of the Hu sheep.

2. A molecular marker related to body weight traits of Hu sheep, wherein ANKRD55 or TRNAW-CCA-87 is the molecular marker related to the body weight traits of the Hu sheep.
